(19) United States
(12) Patent Application Publication
(10) Pub. No.: US 2008/0286464 A1
(43) Pub. Date: Nov. 20, 2008

(54) Group 2 Metal Precursors For Depositing Multi-Component Metal Oxide Films

(75) Inventors: Xinjian Lei, Vista, CA (US); Liam J. Quinn, Taipei (TW); Daniel P. Spence, San Diego, CA (US)

Correspondence Address: AIR PRODUCTS AND CHEMICALS, INC.;PATENT DEPARTMENT, 7201 HAMILTON BOULEVARD, ALLENTOWN, PA, 181951501, US

(73) Assignee: AIR PRODUCTS AND CHEMICALS, INC., Allentown, PA (US)

(21) Appl. No.: 12/113,397

(22) Filed: May 1, 2008

Related U.S. Application Data
(60) Provisional application No. 60/938,233, filed on May 16, 2007.

(51) Int. Cl. C23C 16/18; C07C 43/02; C07C 233/00

(52) U.S. Cl. 427/255.394; 568/579; 564/123

(57) ABSTRACT

Novel Sr and Ba complexes containing both beta-diketonates and N-methyl-pyrrolidone were synthesized and characterized. TGA experiments indicated these complexes are volatile, they can be employed as potential precursors for ALD strontium titanate (STO) or barium strontium titanate films (BST) films in semiconductor fabrication.

### CROSS REFERENCE TO RELATED APPLICATIONS

This patent application claims the benefit of U.S. Provisional Patent Application Ser. No. 60/938,233 filed 16 May 2007.

### BACKGROUND OF THE INVENTION

The semiconductor fabrication industry has an interest in depositing barium strontium titanate films (“BST”) for constructing various electronic devices in integrated circuits. The industry has sought precursors of barium strontium and titanium that are stable, liquid and readily decompose under standard deposition conditions leaving high purity BST films and removing in the vapor phase the remainder of the precursors of barium, strontium and titanium, typically in the form of ligands that reversibly bind the metals until the deposition conditions are provide wherein the metal is deposited from the ligand precursor resulting in essentially deposited metal and volatile, gas phase leaving groups constituting the ligand that bound the metal in the precursor form or decomposition components of the ligand which still exhibit the properties of being good leaving groups that do not contaminate the deposited BST film and typically leave as volatile, gaseous groups.

Representative art to this field includes:
Auld, J.; Jones, A. C.; Leese, A. B.; Cockayne, B.; Wright, P. J.; O'Brien, P.; Motevalli, M., “Vapor-phase transport of barium b-diketonates in the deposition of oxides and the isolation and structural characterization of a novel hexameric cluster of 2,2,6,6-tetramethylheptane-3,5-dionato-barium(II)”, Journal of Materials Chemistry, 3, (12), 1203-8, (1993).
Baum, T. H., “Alkane and polyamine solvent compositions for liquid delivery chemical vapor deposition”, 99-454954 6344079, 19991203, (2002).
Baum, T. H.; Paw, W., “Group IIA β-diketonate tetrahydrofuran-adduct complexes as source reagents for chemical vapor deposition”, 99-321637 6218518, 19990528, (2001).
Bedoya, C.; Condorelli, G. G.; Motta, A.; Mauro, A. D.; Anastasi, G.; Fragalà, I. L.; Lisoni, J. G.; Wouters, D., “MOCVD of Sr-Containing Oxides: Transport Properties and Deposition Mechanisms of the Sr(tmhd)₂.pmdeta Precursor”, Chemical Vapor Deposition, 11, 269-275, (2005).
Brooks, J.; Davies, H. O.; Leedham, T. J.; Jones, A. C.; Steiner, A., “The crystal structure of unadducted strontium bis-tetramethylheptanedionate: The standard precursor for the MOCVD of strontium-containing oxides”, Chemical Vapor Deposition, 6, (2), 66+, (2000).
Cheol Seong, H.; Jaehoo, P.; Doo Sup, H.; Cha Young, Y., Compositional Variation of Metallorganic Chemically Vapor Deposited SrTiO₃ Thin Films along the Capacitor Hole Having a Diameter of 0.15 mu m. In ECS: (2001); Vol. 148, pp G636-G639.
Drake, S. R.; Miller, S. A. S.; Williams, D. J., “Monomeric Group IIA metal β-diketonates stabilized by multidentate glymes”, Inorganic Chemistry, 32, (15), 3227-35, (1993).
Drozdov, A. A.; Trojanov, S. I., “New oligomeric structures of barium dipivaloylmethanate, Ba₄(tmhd)₈, and its pivalate derivative Ba₅(tmhd)₉(piv)”, Polyhedron 11, 2877-2882 (1992).
Gardiner, R. A.; Gordon, D. C.; Stauf, G. T.; Vaartstra, B. A., “Mononuclear Barium Diketonate Polyamine Adducts—Synthesis, Structures, and Use in MOCVD of Barium-Titanate”, Chemistry of Materials, 6, (11), 1967-1970, (1994).
Ghandari, G.; Glassman, T. E.; Studebaker, D. B.; Stauf, G.; Baum, T. H., “Comparison of (L)M(tmhd)₂(M=Mg, Ca, Sr, Ba; L=tetraglyme, pmdeta) precursors for high K dielectric MOCVD”, Materials Research Society Symposium Proceedings, 446, (Amorphous and Crystalline Insulating Thin Films—1996), 327-332, (1997).
Glassman, T. E.; Bhandari, G.; Baum, T. H., “Evidence for cooperative oxidation of MOCVD precursors used in BaₓSr₁₋ₓTiO₃ film growth”, Materials Research Society Symposium Proceedings, 446, (Amorphous and Crystalline Insulating Thin Films—1996), 321-326, (1997).
Hintermaier, F. S.; Baum, T. H., “Alkane and polyamine solvent compositions for liquid delivery chemical vapor deposition”, 98-185374 6214105, 19981103, (2001).
Hubertpfalzgraf, L. G.; Labrize, F., “Barium Beta-Diketonate Derivatives with Aminoalcohols—Synthesis, Molecular-Structure and Thermal-Behavior of Ba₅(μ₄-OH)(μ₃, η²-OCH(Me)CH₂NMe₂)₄(μ, η² tmhd)₄(η²-tmhd) and of [Ba(μ,η²:η²-tmhd)(η²-tmhd)(η²-OHCH(Me)CH₂NMe₂)]₂”, Polyhedron, 13, (14), 2163-2172, (1994).
Hwang, C. S.; No, S. Y.; Park, J.; Kim, H. J.; Cho, H. J.; Han, Y. K.; Oh, K. Y., “Cation Composition Control of MOCVD (Ba,Sr)TiO₃ Thin Films along the Capacitor Hole”, Journal of The Electrochemical Society, 149, (10), G585-G592, (2002).
Kwon, O, S.; Kim, S. K.; Cho, M.; Hwang, C. S.; Jeong, J., “Chemically Conformal ALD of SrTiO₃ Thin Films Using Conventional Metallorganic Precursors”, Journal of The Electrochemical Society, 152, (4), C229-C236, (2005).
Kwon, O, S.; Lee, S. W.; Han, J. H.; Hwang, C. S., “Atomic Layer Deposition and Electrical Properties of SrTiO₃ Thin Films Grown Using Sr(C₁₁H₁₉O₂)₂, Ti(O₁—C₃H₇)₄, and H₂O”, J. Electrochem. Soc., 154, G127-G133, (2007).
Min, Y.-S.; Cho, Y. J.; Kim, D.; Lee, J.-H.; Kim, B. M.; Lim, S. K.; Lee, I. M.; Lee, W. I., “Liquid source-MOCVD of BaₓSr₁₋ₓTiO₃ (BST) thin films with a N-alkoxy-b-ketoiminato titanium complex”, Chemical Vapor Deposition, 7, (4), 146-149, (2001).
Ohkawa, A.; Tsutsumi, Y., “Deposition of thin films in fabrication of semiconductor devices by MOCVD process”, 98-263702 2000100802, 19980917, (2000).
Paw, W.; Baum, T. H., “Preparation of Group IIA metal b-diketonate Lewis base MOCVD source reagents, and method of forming Group IIA metal-containing films utilizing same”, 6338873, Jul. 6, 2000, (2002).
Roeder, J. F.; Stauf, G. T., “Liquid delivery MOCVD process for deposition of high frequency dielectric materials”, 99-US29566 2000037712, 19991213, (2000).
Rossetto, G.; Polo, A.; Benetollo, F.; Porchia, M.; Zanella, P., “Studies on Molecular Barium Precursors for Mocvd—Synthesis and Characterization of Barium 2,2,6,6-Tetramethyl-3,5-Heptanedionate —X-Ray Crystal-Structure of [Ba(tmhd)₂.Et₂O]₂”, Polyhedron, 11, (8), 979-985, (1992).
Stauf, G. T.; Roeder, J. F.; Baum, T. H., “Liquid delivery MOCVD process for deposition of high frequency dielectric materials”, 98-216673 6277436, 19981218, (2001).

### BRIEF SUMMARY OF THE INVENTION

The present invention is directed to a metal containing complex represented by the structures selected from the group consisting of:

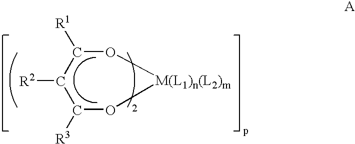

wherein M is selected from Mg, Ca, Sr, and Ba; R¹ and R³ are independently selected from the group consisting of linear or branched alkyl, alkoxyalkyl, fluoroalkyl: having from 1 to 10 carbon atoms, cycloaliphatic, and aryl having from 4 to 12 carbon atoms; R² is selected from the group consisting of hydrogen, linear or branched alkyl, fluoroalkyl, alkoxy: having from 1 to 10 carbon atoms, cycloaliphatic, and aryl: having from 4 to 12 carbon atoms; L₁ is selected from the group consisting of the organic amide class RCONR′R″ wherein R and R′ are linear or branched alkyl having from 1-10 carbon atoms and they can be connected to form a cyclic group (CH₂)_{q}, wherein q is from 4-6, and R″ is selected from alkyl having from 1 to 4 carbon atoms and cycloalkyl having from 4 to 8 carbon atoms; L₂ is selected from the group consisting of H₂O and ROH wherein R is linear or branched alkyl having from 1-10 carbon atoms and they can be connected to form a cyclic group (CH₂)_{q}, wherein q is from 4-6; n is a number selected from between 1 and 4; m is selected from a number between 0 to 4, p is selected from 1, 2;

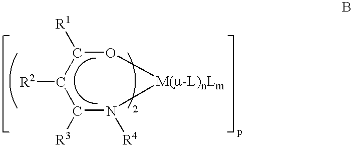

wherein M is selected from Mg, Ca, Sr, and Ba; R¹ and R⁴ are selected from the group consisting of linear or branched alkyl, alkoxyalkyl, fluoroalkyl: having from 1 to 10 carbon atoms, cycloaliphatic, and aryl: having from 4 to 12 carbon atoms; R² and R³ are selected from the group consisting of hydrogen, linear or branched alkyl, alkoxyalkyl, fluoroalkyl, alkoxy: having from 1 to 10 carbon atoms, cycloaliphatic, and aryl: having from 4 to 12 carbon atoms, L is selected from the group of the organic amide class RCONR′R″ wherein R and R′ are independently alkyl having from 1-10 carbon atoms and they can be connected to form a cyclic group (CH₂)_{q}, wherein q is from 4-6, and R″ is selected from alkyl having from 1 to 4 carbon atoms and cycloalkyl having from 4 to 8 carbon atoms; n is a number selected from between 0 and 4; m is selected from a number between 1 to 4; p is selected from 1, 2; μ-L indicates L is connected to two metals, M, via μL's oxygen atom when p=2; and,

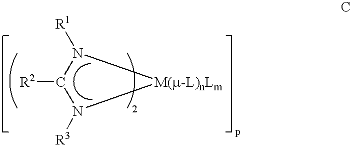

wherein M is selected from Mg, Ca, Sr, and Ba; R¹ and R³ are independently selected from the group consisting of linear or branched alkyl, alkoxyalkyl, fluoroalkyl: from 1 to 10 carbon atoms, cycloaliphatic, and aryl: having from 4 to 12 carbon atoms; R² is selected from the group consisting of hydrogen, linear or branched alkyl, fluoroalkyl, alkoxy: from 1 to 10 carbon atoms, cycloaliphatic, and aryl: having from 4 to 12 carbon atoms; L is selected from the group of the organic amide class RCONR′R″ wherein R and R′ are independently linear or branched alkyl having from 1-10 carbon atoms and they can be connected to form a cyclic group (CH₂)_{q}, wherein q is from 4-6, and R″ is selected from alkyl having from 1 to 4 carbon atoms and cycloalkyl: having from 4 to 8 carbon atoms, n is a number selected from between 0 and 4; m is selected from a number between 1 to 4; p is selected from 1, 2; μ-L indicates L is connected to two metals, M, via μL's oxygen atom when p=2.

### BRIEF DESCRIPTION OF THE DRAWINGS

FIG. 1 is a drawing representative of the crystal structure of Sr₂(tmhd)₄(NMP)₄.

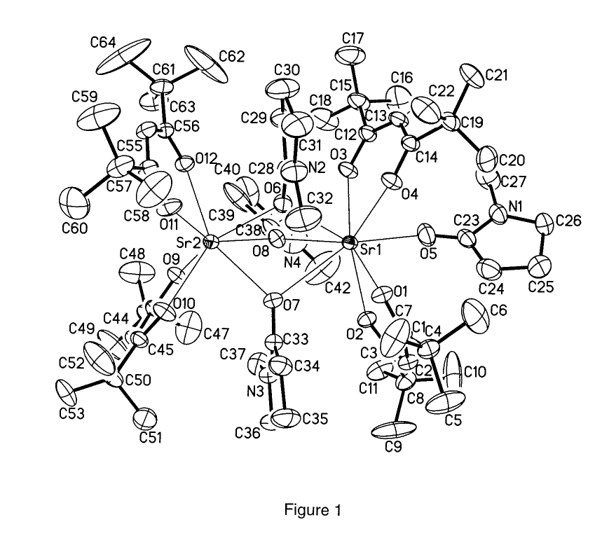

FIG. 2 is a drawing representative of the crystal structure of Sr₂(tmhd)₄(DEAC)₃.

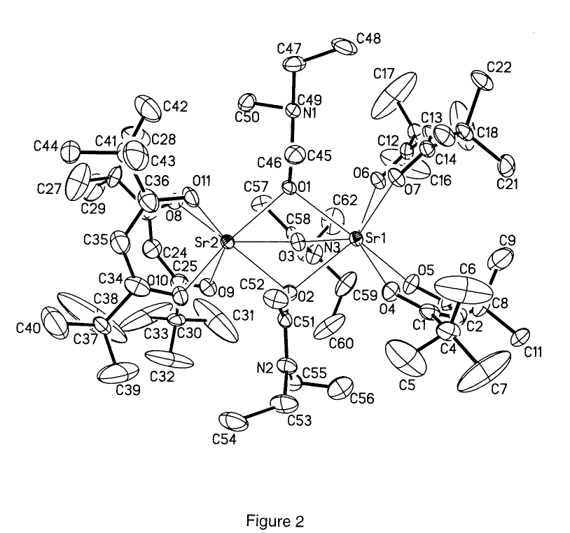

FIG. 3 is TGAs of Sr₂(tmhd)₄(NMP)₄ (dashed line) and Sr₂(tmhd)₄(DEAC)₃ (solid line), showing both are volatile complexes.

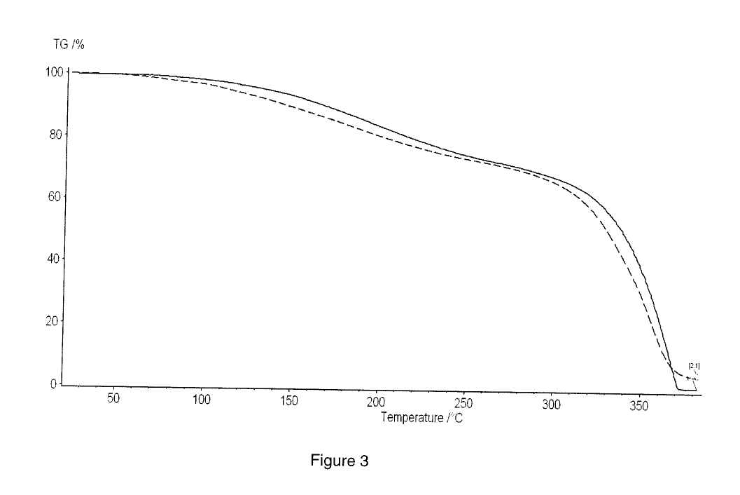

FIG. 4 is a drawing representative of the crystal structure of [Ba(tmhd)₂(NMP)₂.H₂O]₂.

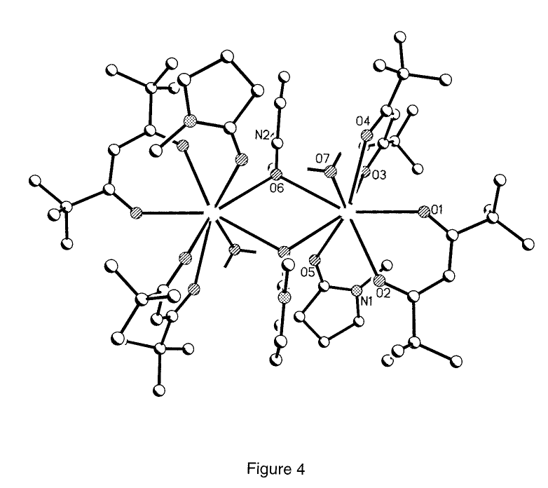

FIG. 5 is TGA of [Ba(tmhd)₂(NMP)₂.H₂O]₂, indicating it is volatile.

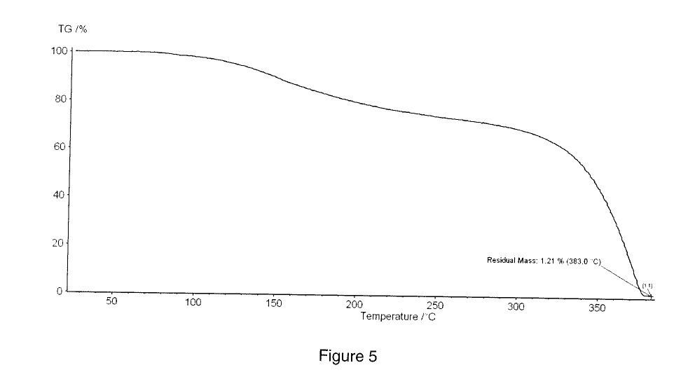

### DETAILED DESCRIPTION OF THE INVENTION

Novel Sr and Ba complexes containing both beta-diketonates and N-methyl-pyrrolidone have been synthesized and characterized. Thermogravimetric Analysis (TGA) experiments indicate these complexes are volatile, and that they can be employed as potential precursors for chemical vapor deposition (CVD), cyclic chemical vapor deposition (CCVD), or atomic layer deposition (ALD) of strontium titanate (STO) or barium strontium titanate films (BST) films in semiconductor fabrication. In the deposition of STO and BST films, the titanium source is selected from titanium containing precursors exemplified by titanium alkoxides or beta-diketonates such as Ti(OPrⁱ)₄, Ti(tmhd)₂(OPrⁱ)₂, where Prⁱ=isopropyl, where tmhd=2,2,6,6-tetramethyl-3,5-heptanedionate, Ti(mpd)(tmhd)₂, where mpd=2-methyl-2,4-pentanedioxy, Ti(4-(2-methylethoxy)imino-2-pentanoate)₂, and analogous titanium ligands and derivatives.

These Group 2 metal complexes are precursors capable of depositing Group 2 metal-containing films for semiconductor applications. The metal complexes include:

(i) Group 2 beta-diketonate with organic amides as adducts with a formula of [M(R¹C(O)CR²C(O)R³)₂(L₁)ₙ(L₂)ₘ]ₚ wherein R¹ and R³ are independently selected from the group consisting of linear or branched alkyl, alkoxyalkyl, fluoroalkyl: having from 1 to 10 carbon atoms, cycloaliphatic, and aryl: having from 4 to 12 carbon atoms; R² is selected from the group consisting of hydrogen, linear or branched alkyl, fluoroalkyl, alkoxy: having from 1 to 10 carbon atoms, cycloaliphatic, and aryl: having from 4 to 12 carbon atoms; L, is selected from the group consisting of the organic amide class RCONR′R″ wherein R and R′ are linear or branched alkyl having from 1-10 carbon atoms and they can be connected to form a cyclic group (CH₂)_{q}, wherein q is from 4-6, and R″ is selected from alkyl having from 1 to 4 carbon atoms and cycloalkyl having from 4 to 8 carbon atoms; L₂ is selected from the group consisting of the H₂O and ROH wherein R is linear or branched alkyl having from 1-10 carbon atoms and they can be connected to form a cyclic group (CH₂)_{q}, wherein q is from 4-6; n is a number selected from between 1 and 4; m is selected from a number between 0 to 4; p is selected from 1, 2; and,

(ii) Group 2 beta-ketoiminate with organic amides as adducts with a formula of [M(R¹C(O)CR²C(NR³)R⁴)₂(μ-L)ₙLₘ]ₚ wherein R¹ and R⁴ are independently selected from the group consisting of alkyl, alkoxyalkyl, fluoroalkyl: having from 1 to 10 carbon atoms, cycloaliphatic, and aryl: having from 4 to 12 carbon atoms; R² and R³ are independently selected from the group consisting of hydrogen, alkyl, alkoxyalkyl, fluoroalkyl, alkoxy: having from 1 to 10 carbon atoms, cycloaliphatic, and aryl: having from 4 to 12 carbon atoms; L is selected from the group of the organic amide class of the form RCONR′R″ wherein R and R′ are independently alkyl having from 1-10 carbon atoms and they can be connected to form a cyclic group (CH₂)_{q}, wherein q is from 4-6, preferably 5, and R″ is selected from alkyl having from 1 to 4 carbon atoms and cycloalkyl having from 4 to 8 carbon atoms; n is a number selected from between 1 and 10, preferably 3, 4; m is selected from a number between 0 to 4; and p is selected from 1, 2; p-L indicates L is connected to two metals, M, via μL's oxygen atom when p=2.

(iii) Group 2 amidinates with organic amides as adducts with a formula of [M(R¹NC(R²)NR³)₂(μ-L)ₙLₘ]ₚ wherein R¹ and R³ are independently selected from the group consisting of linear or branched alkyl, alkoxyalkyl, fluoroalkyl: from 1 to 10 carbon atoms, cycloaliphatic, and aryl: having from 4 to 12 carbon atoms; R² is selected from the group consisting of hydrogen, linear or branched alkyl, fluoroalkyl, alkoxy: from 1 to 10 carbon atoms, cycloaliphatic, and aryl: having from 4 to 12 carbon atoms; L either bridging between two metal atoms or coordinating to one metal atom is selected from the group of the organic amide class RCONR′R″ wherein R and R′ are independently linear or branched alkyl having from 1-10 carbon atoms and they can be connected to form a cyclic group (CH₂)_{q}, wherein q is from 4-6, and R″ is selected from alkyl having from 1 to 4 carbon atoms and cycloalkyl: having from 4 to 8 carbon atoms, n is a number selected from between 0 and 4; m is selected from a number between 1 to 4; and p is selected from 1, 2; μ-L indicates L is connected to two metals, M, via μL's oxygen atom when p=2.

This invention is related to Group 2 metal-containing complexes having both beta-ketonate or beta-ketoiminate or amidinate and organic amides and their solutions, which are useful for fabricating conformal metal containing films on substrates such as silicon, metal nitride, metal oxide and other metal layers via deposition processes, e.g., chemical vapor deposition (CVD), plasma enhanced chemical vapor deposition (PECVD), low pressure chemical vapor deposition (LPCVD), plasma enhanced atomic layer deposition (PE ALD) and atomic layer deposition (ALD). Such conformal metal containing films have applications ranging from computer chips, optical device, magnetic information storage, to metallic catalyst coated on a supporting material. In contrast to prior polydentate beta-ketoiminate precursors, the polydentate beta-ketoiminate ligands incorporate at least one amino organo imino functionality, which is in contrast to the literatures reported alkoxy group as the donating ligand.

One type of structure in the metal precursor is illustrated in structure A below where the metal M is a Group 2 metal having the formula:

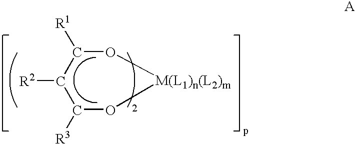

wherein M is selected from Mg, Ca, Sr, and Ba; R¹ and R³ are independently selected from the group consisting of linear or branched alkyl, alkoxyalkyl, fluoroalkyl: having from 1 to 10 carbon atoms, cycloaliphatic, and aryl having from 4 to 12 carbon atoms; R² is selected from the group consisting of hydrogen, linear or branched alkyl, fluoroalkyl, alkoxy: having from 1 to 10 carbon atoms, cycloaliphatic, and aryl: having from 4 to 12 carbon atoms; L₁ is selected from the group consisting of the organic amide class RCONR′R″ wherein R and R′ are linear or branched alkyl having from 1-10 carbon atoms and they can be connected to form a cyclic group (CH₂)_{q}, wherein q is from 4-6, and R″ is selected from alkyl having from 1 to 4 carbon atoms and cycloalkyl having from 4 to 8 carbon atoms; L₂ is selected from the group consisting of the organic amide class RCONR′R″ wherein R and R′ are independently linear or branched alkyl having from 1-10 carbon atoms and they can be connected to form a cyclic group (CH₂)_{q}, wherein q is from 4-6 and neutral oxygen; n is a number selected from between 1 and 4; m is selected from a number between 0 to 4; p is selected from 1, 2.

Another type of structure in the metal precursor is illustrated in structure B below where the metal M is a Group 2 metal having the formula:

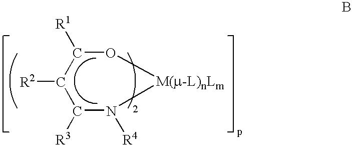

wherein M is selected from Mg, Ca, Sr, and Ba; R¹ and R⁴ are selected from the group consisting of linear or branched alkyl, alkoxyalkyl, fluoroalkyl: having from 1 to 10 carbon atoms, cycloaliphatic, and aryl: having from 4 to 12 carbon atoms; R² and R³ are selected from the group consisting of hydrogen, linear or branched alkyl, alkoxyalkyl, fluoroalkyl, alkoxy: having from 1 to 10 carbon atoms, cycloaliphatic, and aryl: having from 4 to 12 carbon atoms, L is selected from the group of the organic amide class RCONR′R″ wherein R and R′ are independently alkyl having from 1-10 carbon atoms and they can be connected to form a cyclic group (CH₂)_{q}, wherein q is from 4-6, and R″ is selected from alkyl having from 1 to 4 carbon atoms and cycloalkyl having from 4 to 8 carbon atoms; n is a number selected from between 0 and 4; m is selected from a number between 1 to 4; p is selected from 1, 2; μ-L indicates L is connected to two metals, M, via μL's oxygen atom when p=2.

The third type of structure in the metal precursor is illustrated in structure C below where the metal M is a Group 2 metal having the formula:

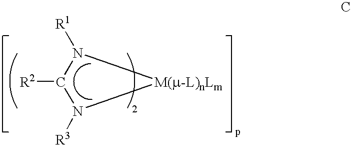

wherein M is selected from Mg, Ca, Sr, and Ba; R¹ and R³ are independently selected from the group consisting of linear or branched alkyl, alkoxyalkyl, fluoroalkyl: from 1 to 10 carbon atoms, cycloaliphatic, and aryl: having from 4 to 12 carbon atoms; R² is selected from the group consisting of hydrogen, linear or branched alkyl, fluoroalkyl, alkoxy: from 1 to 10 carbon atoms, cycloaliphatic, and aryl: having from 4 to 12 carbon atoms; L is selected from the group of the organic amide class RCONR′R″ wherein R and R′ are independently linear or branched alkyl having from 1-10 carbon atoms and they can be connected to form a cyclic group (CH₂)_{q}, wherein q is from 4-6, and R″ is selected from alkyl having from 1 to 4 carbon atoms and cycloalkyl: having from 4 to 8 carbon atoms; n is a number selected from between 0 and 4; m is selected from a number between 1 to 4; p is selected from 1, 2; μ-L indicates L is connected to two metals, M, via μL's oxygen atom when p=2.

These metal-containing complexes having both beta-ketonate or beta-ketoiminate ligands and organic amides can be employed as potential precursors to make thin metal or metal oxide films via either the chemical vapor deposition (CVD) or atomic layer deposition (ALD) method at temperatures less than 500° C. The CVD process can be carried out with or without reducing or oxidizing agents, whereas an ALD process usually involves the employment of another reactant, such as a reducing agent or oxidizing agent.

For multi-component metal oxides such as STO and BST, these metal-containing complexes, having beta-ketonate or beta-ketoiminate or amidinate ligands and organic amides, can be delivered in vapor phase into a CVD or ALD reactor via well-known bubbling or vapor draw techniques as strontium or barium sources. The titanium source is selected from titanium alkoxides or beta-diketonates such as Ti(OPrⁱ)₄, Ti(tmhd)₂(OPrⁱ)₂, where Prⁱ=isopropyl, where tmhd=2,2,6,6-tetramethyl-3,5-heptanedionate, Ti(mpd)(tmhd)₂, where mpd=2-methyl-2,4-pentanedioxy, Ti(4-(2-methylethoxy)imino-2-pentanoate)₂, and analogous titanium ligands and derivatives. A direct liquid delivery method can also be employed by dissolving the titanium, strontium as well as barium complexes in a suitable solvent or a solvent mixture to prepare a solution with a molar concentration from 0.001 to 2 M, depending the solvent or mixed-solvents employed. Oxidizing agents for the deposition process include oxygen, water, hydrogen peroxide, oxygen plasma, nitrous oxide, and ozone. In a preferred embodiment, the multi-compoent metal oxide films are grown in the temperature range of 200 to 500° C., preferably 250 to 350° C. whereby an amorphous STO or BST films are obtained. A thermal annealing is needed to convert the resulting films from amorphous into crystalline form. The annealing can be conducted at a higher temperature 500 to 1200° C. under oxidizing conditions, preferably in the range of 500 to 700° C. for high k dielectrics in DRAM applications. The thickness of the STO or BST film is in the range of 1 nm to 500 nm, preferably 2 nm to 10 nm, deposited on compatible substrates including platinum(Pt), RuO₂, SrRuO₃, silica, silicon nitride, and silicon. The process chamber pressure may preferably be from about 0.1 Torr to 100 Torr, and more preferably from about 0.1 Torr to 5 Torr.

The solvent employed in solubilizing the precursor for use in a deposition process may comprise any compatible solvent or their mixture, including: aliphatic hydrocarbons, aromatic hydrocarbons, ethers, esters, nitrites, and alcohols. The solvent component of the solution preferably comprises a solvent selected from the group consisting of: glyme solvents having from 1 to 20 ethoxy —(C₂H₄O)— repeat units; C₂-C₁₂ alkanols, organic ethers selected from the group consisting of dialkyl ethers comprising C₁-C₆ alkyl moieties, C₄-C₈ cyclic ethers; C₁₂-C₆₀ crown O₄-O₂₀ ethers (wherein C is the number of carbon atoms in the ether compound and O is the number of oxygen atoms in the ether compound); C₆-C₁₂ aliphatic hydrocarbons; C₆-C₁₈ aromatic hydrocarbons; organic esters; organic amines, polyamines and organic amides.

Another class of solvents that offers advantages is the organic amide class of the form RCONR′R″, wherein R and R′ are independently linear or branched alkyl having from 1-10 carbon atoms, and they can be connected to form a cyclic group (CH₂)_{q}, wherein q is from 4-6, preferably 5, and R″ is selected from alkyl having from 1 to 4 carbon atoms and cycloalkyl having 4 to 8 carbon atoms. N-methylpyrrolidinone (NMP), N,N-Diethylformamide (DEF), N,N-Diethylacetamide (DEAC), N,N-Dimethylacetamide (DMAC), and N-cyclohexyl 2-pyrrolidinone are examples.

The following examples illustrate the preparation of the metal-containing complexes with beta-diketone or beta-ketoiminate ligands as well as their use as precursors in metal-containing film deposition processes.

### EXAMPLE 1

### Synthesis of Ba₂(hfac)₄(NMP)₅

0.52 g (3.80 mmol) of BaH₂ was loaded in flask with 15 mL of toluene. To this flask was added 2.15 g (21.70 mmol) NMP followed by addition of 1.57 g (7.60 mmol) 1,1,1,5,5,5-hexafluoro-2,4-pentanedione (“hfac”). Stirred reaction mixture at room temperature until bubbling ceased after 5-10 minutes. Removed volatiles under vacuum and noticed the formation of a white solid. Upon completion of evaporation, a liquid was left which was taken up in hexane and recrystallized in −40° C. freezer. Decanted out hexanes from recrystallized solid and solid taken up in toluene and recrystallized again in freezer. Decanted toluene from crystals. Crystals identified by crystal structure analysis as the dimer Ba₂(hfac)₄(NMP)₅.

### EXAMPLE 2

### Synthesis of Sr₂(tmhd)₄(NMP)₄

0.50 g (5.58 mmol) of SrH₂ was loaded into a 100 mL Schlenk flask with 5 g of NMP. To this flask was added 2.06 g (11.16 mmol) 2,2,6,6-tetramethyl-3,5-heptanedione (“tmhd”) in 5 g of NMP and bubbling occurred. Left to stir over night at room temperature. Reaction mixture was subjected to vacuum transferred at 65-70° C. leaving behind a grey solid weighing 2.86 g. Recrystallized in a 1:1 solution of hexane to pentane in −40° C. freezer to afford clear needle like crystals that were identified by crystal structure analysis as the dimer Sr₂(tmhd)₄(NMP)₄.

### EXAMPLE 3

### Synthesis of Sr₂(tmhd)₄(DEAC)₃

To a suspension of 0.50 g (5.58 mmol) SrH₂ in hexanes was added 20.10 g (11.16 mmol) 2,2,6,6-tetramethyl-3,5-heptanedione (“tmhd”) and 2.60 g (22.32 mmol) N,N-diethylacetamide (DEAC) in hexanes at room temperature. Bubbling and heat were given off and the suspension turned to solution after approximately 10 minutes. The reactin mixture was stirred for 16 hours and removal of hexanes to afford an oil that was set up for vacuum transfer and heated at 80° C. under 300 mTorr for several hours. 2.38 g of clear residual slurry (68% yield) was collected and recrystallization in pentane an −40° C. gave rise to clear crystals. The crystals were identified by crystal structure analysis as Sr₂(tmhd)₄(DEAC)₃

### EXAMPLE 4

### Synthesis of Ba₂(tmhd)₄(NMP)₄(H₂O)₂

0.50 g (3.59 mmol) of BaH₂ was loaded into a 100 mL Schlenk flask with 15 mL of hexane. To this flask was added a solution of 1.32 g (7.18 mmol) tmhd and 1.42 (14.35 mmol) NMP in 5 mL hexane and bubbling was witnessed. After approximately 6 hours, the hexane was evaporated under vacuum leaving behind a grey solid weighing 2.32 g. Recrystallized in octane giving clear hexagonal-like crystals that were sent for crystal structure analysis and confirmed as the proposed dimer. A 90% yield was obtained based off of crude.

### EXAMPLE 5

### Synthesis of 2,2-dimethyl-5-(iso-propylamino)-3-hexanone

To a solution of 15.00 g (105.49 mmol) 2,2-dimethyl-3,5-hexanedione in 50 mL of toluene loaded with 30.00 g sodium sulfate was added 12.47 g (210.97 mmol) isopropylamine. The mixture was refluxed for 4 days. Removal of toluene resulted in a yellow oil, which was subjected to vacuum transfer at 80° C. under 125 mTorr. 16.5 g of clear oil was obtained with a yield of 84%. ¹H NMR (500 MHz, C₆D₆): δ=11.50 (s, 1H), 5.16 (s, 1H), 3.11 (m, 1H), 1.49 (s, 3H), 1.31 (s, 9H), 0.79 (d, 6H).

### EXAMPLE 6

### Synthesis of 2,2-dimethyl-5-(sec-butylamino)-3-hexanone

To a solution of 5.00 g (35.16 mmol) 2,2-dimethyl-3,5-hexanedione in 20 mL of toluene loaded with 10.00 g of sodium sulfate was added 5.14 g (70.32 mmol) sec-butylamine. The mixture was refluxed 3 days. 4.90 g of light yellow oil was obtained after work-up. The isolated yield was 71%. ¹H NMR (500 MHz, C₆D₆): δ=11.52 (s, 1H), 5.18 (s, 1H), 2.95 (m, 1H), 1.51 (s, 3H), 1.31 (s, 9H), 1.14 (s, 2H), 0.80 (d, 3H), 0.67 (t, 3H).

### EXAMPLE 7

### Synthesis of bis(2,2-dimethyl-5-(iso-propylamino)-3-hexanonato)barium NMP adduct

To a suspension of 1 g (1.66 mmol) Ba(N(SiMe₃)₂)₂(THF)₂ in hexane was added 0.61 g (3.32 mmol) of 2,2-dimethyl-5-(iso-propylamino)-3-hexanone and 0.66 g (6.64 mmol) of NMP in hexane at room temperature. The reaction mixture turned to solution after approximately 30 minutes. After tirring for 16 hours, hexane was evaporated under vacuum to provide 0.95 g of a white solid. The white solid was heated in hexane, filtered, and recrystallized an −40° C. to give rise to foggy-white crystals. ¹H NMR (500 MHz, C₆D₆): δ=5.06 (s, 1H), 3.75 (m, 1H), 2.42 (s, 3H), 2.40 (t, 2H), 1.98 (t, 2H), 1.83 (s, 3H), 1.44 (s, 9H), 1.34 (d, 6H), 1.17 (m, 2H).

### EXAMPLE 8

### Synthesis of bis(2,2-dimethyl-5-(sec-butylamino)-3-hexanonato)strontium NMP adduct

To a solution of 1 g (1.81 mmol) of Sr(N(SiMe₃)₂)₂(THF)₂ in hexanes was added 0.66 g (3.62 mmol) of 2,2-dimethyl-5-(sec-butylamino)-3-hexanone and 0.72 g (7.24 mmol) of NMP in hexanes at room temperature. After stirring for 16 hours, hexanes were evaporated under vacuum. Recrystallization in hexanes at −20° C. resulted in a opaque white solid. ¹H NMR (500 MHz, C₆D₆): δ=5.06 (s, 1H), 3.75 (m, 1H), 2.46 (s, 3H), 2.42 (t, 2H), 2.07 (t, 2H), 1.90 (s, 3H), 1.47 (s, 9H), 1.47 (d, 6H), 1.21 (m, 2H).

### EXAMPLE 9

### Synthesis of bis(2,2-dimethyl-5-(sec-butylamino)-3-hexanonato)strontium DEAC adduct

To a solution of 1 g (1.81 mmol) Sr(N(SiMe₃)₂)₂(THF)₂ in 10 mL hexanes at room temperature was added 0.71 g (3.62 mmol) of 2,2-dimethyl-5-(sec-butylamino)-3-hexanone and 0.83 g (7.24 mmol) DEAC in 10 mL hexanes. Reaction was stirring for 16 hours. Evaporation of hexanes afforded a wet off-white solid. ¹H NMR (500 MHz, C₆D₆): δ=5.05 (s, 1H), 3.59 (m, 1H), 3.17 (q, 2H), 2.51 (q, 2H), 1.96 (s, 3H), 1.79 (s, 3H), 1.55 (s, 9H) 1.44 (m, 2H), 1.44 (d, 3H), 1.03 (t, 3H) 0.96 (t, 3H), 0.56 (t, 3H).

1. A metal containing complex represented by the structure selected from the group consisting of: 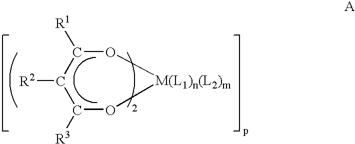
wherein M is selected from Mg, Ca, Sr, and Ba; R¹ and R³ are independently selected from the group consisting of linear or branched alkyl, alkoxyalkyl, fluoroalkyl: having from 1 to 10 carbon atoms, cycloaliphatic, and aryl having from 4 to 12 carbon atoms; R² is selected from the group consisting of hydrogen, linear or branched alkyl, fluoroalkyl, alkoxy: having from 1 to 10 carbon atoms, cycloaliphatic, and aryl: having from 4 to 12 carbon atoms; L₁ is selected from the group consisting of the organic amide class RCONR′R″ wherein R and R′ are linear or branched alkyl having from 1-10 carbon atoms and they can be connected to form a cyclic group (CH₂)_{q}, wherein q is from 4-6, and R″ is selected from alkyl having from 1 to 4 carbon atoms and cycloalkyl having from 4 to 8 carbon atoms; L₂ is selected from the group consisting of the organic amide class RCONR′R″ wherein R and R′ are independently linear or branched alkyl having from 1-10 carbon atoms and they can be connected to form a cyclic group (CH₂)_{q}, wherein q is from 4-6; n is a number selected from between 1 and 4; m is selected from a number between 0 to 4; p is selected from 1, 2; 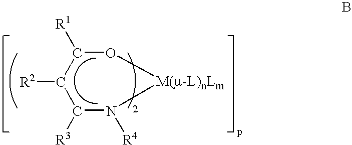
wherein M is selected from Mg, Ca, Sr, and Ba; R¹ and R⁴ are selected from the group consisting of linear or branched alkyl, alkoxyalkyl, fluoroalkyl: having from 1 to 10 carbon atoms, cycloaliphatic, and aryl: having from 4 to 12 carbon atoms; R² and R³ are selected from the group consisting of hydrogen, linear or branched alkyl, alkoxyalkyl, fluoroalkyl, alkoxy: having from 1 to 10 carbon atoms, cycloaliphatic, and aryl: having from 4 to 12 carbon atoms, L is selected from the group of the organic amide class RCONR′R″ wherein R and R′ are independently alkyl having from 1-10 carbon atoms and they can be connected to form a cyclic group (CH₂)_{q}, wherein q is from 4-6, and R″ is selected from alkyl having from 1 to 4 carbon atoms and cycloalkyl having from 4 to 8 carbon atoms; n is a number selected from between 0 and 4; m is selected from a number between 1 to 4; p is selected from 1, 2; p-L indicates L is connected to two metals, M, via μL's oxygen atom when p=2; and, 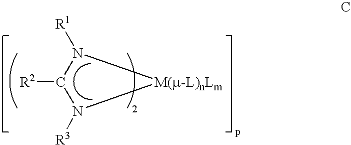
wherein M is selected from Mg, Ca, Sr, and Ba; R¹ and R³ are independently selected from the group consisting of linear or branched alkyl, alkoxyalkyl, fluoroalkyl: from 1 to 10 carbon atoms, cycloaliphatic, and aryl: having from 4 to 12 carbon atoms; R² is selected from the group consisting of hydrogen, linear or branched alkyl, fluoroalkyl, alkoxy: from 1 to 10 carbon atoms, cycloaliphatic, and aryl: having from 4 to 12 carbon atoms; L is selected from the group of the organic amide class RCONR′R″ wherein R and R′ are independently linear or branched alkyl having from 1-10 carbon atoms and they can be connected to form a cyclic group (CH₂)_{q}, wherein q is from 4-6, and R″ is selected from alkyl having from 1 to 4 carbon atoms and cycloalkyl: having from 4 to 8 carbon atoms; n is a number selected from between 0 and 4; m is selected from a number between 1 to 4; p is selected from 1, 2; μ-L indicates L is connected to two metals, M via μL's oxygen atom when p=2.

2. The metal containing complex of claim 1 structure A wherein M is selected from the group consisting of calcium, strontium, barium and mixtures thereof.

3. The metal containing complex of claim 2 wherein L₁ and L₂ are selected from the group consisting of N-methylpyrrolidinone (NMP), N,N-Diethylformamide (DEF), N,N-Diethylacetamide (DEAC), N,N-Dimethylacetamide (DMAC), N-cyclohexyl 2-pyrrolidinone, H₂O, and ROH.

4. The metal containing complex of claim 2 wherein R¹ and R³ are selected from the group consisting of t-butyl and t-pentyl; R² is selected from the group consisting of hydrogen, methyl and ethyl; and R⁴ is selected from the group consisting of iso-propyl, t-butyl, sec-butyl, t-pentyl and mixtures thereof.

5. The metal containing complex of claim 2 wherein M is strontium; R¹ and R³ are each t-butyl, R² is hydrogen, L₁ and L₂ are each N-methyl-pyrrolidone, n=1.5, m=0.5, and p=2.

6. The metal containing complex of claim 2 wherein M is strontium; R¹ and R³ are each t-butyl; R² is hydrogen; L₁ is N,N-Diethylacetamide (DEAC); n=1.5; m=0; and p=2.

7. The metal containing complex of claim 2 wherein M is barium; R¹ and R³ are each CF₃; R² is hydrogen; L₁ and L₂ are each N-methyl-pyrrolidone; n=1.5; m=2; and p=2.

8. The metal containing complex of claim 2 wherein M is barium; R¹ and R³ are each t-butyl; R² is hydrogen; L₁ is N N-methyl-pyrrolidone; n=2; L₂=H₂O; m=1; and p=2.

9. The metal containing complex of claim 1 structure B wherein M is selected from the group consisting of calcium, strontium, and barium.

10. The metal containing complex of claim 9 wherein L is selected from the group consisting of N-methylpyrrolidinone (“NMP”), N,N-Diethylformamide (DEF), N,N-Diethylacetamide (DEAC), N,N-Dimethylacetamide (DMAC), and N-cyclohexyl 2-pyrrolidinone.

11. The metal containing complex of claim 9 wherein R¹ and R³ are independently selected from the group consisting of t-butyl and t-pentyl; R² is selected from the group consisting of hydrogen, methyl and ethyl; and R⁴ is selected from the group consisting of iso-propyl, t-butyl, sec-butyl, t-pentyl and mixtures thereof.

12. The metal containing complex of claim 1 dissolved in a solvent selected from the group consisting of glyme solvents having from 1 to 20 ethoxy —(C₂H₄O)— repeat units; C₂-C₁₂ alkanols, organic ethers; C₆-C₁₂ aliphatic hydrocarbons; C₆-C₁₈ aromatic hydrocarbons; organic esters; organic amines; and polyamines and organic amides.

13. The metal containing complex of claim 12 wherein the solvent is an organic ether selected from the group consisting of dialkyl ethers comprising C₁-C₆ alkyl moieties, C₄-C₈ cyclic ethers; C₁₂-C₆₀ crown O₄-O₂₀ ethers (wherein C is the number of carbon atoms in the crown ether compound and O is the number of oxygen atoms in the crown ether.

14. The metal containing complex of claim 12 wherein the solvent is an organic amide selected from the group consisting of N-methylpyrrolidinone (“NMP”), N,N-Diethylformamide (DEF), N,N-Diethylacetamide (DEAC), N,N-Dimethylacetamide (DMAC), and N-cyclohexyl 2-pyrrolidinone.

15. The metal containing complex of claim 1 structure C wherein M is selected from the group consisting of calcium, strontium, and barium.

16. The metal containing complex of claim 15 wherein R¹ and R³ are independently selected from the group consisting of t-butyl and t-pentyl; R² is selected from the group consisting of hydrogen, methyl and ethyl; and R⁴ is selected from the group consisting of iso-propyl, t-butyl, sec-butyl, t-pentyl and mixtures thereof.

17. A metal containing complex selected from the group consisting of Ba₂(1,1,1,5,5,5-hexafluoro-2,4-pentanedione)₄(N-methylpyrrolidinone)₅, Sr₂(2,2,6,6-tetramethyl-3,5-heptanedione)₄(N-methylpyrrolidinone)₄, Sr₂(2,2,6,6-tetramethyl-3,5-heptanedione)₄(N,N-Diethylacetamide)₃, and Ba₂(2,2,6,6-tetramethyl-3,5-heptanedione)₄(N-methylpyrrolidinone)₄.(H₂O)₂, bis(2,2-dimethyl-5-(iso-propylamino)-3-hexanonato)barium(N-methylpyrrolidinone), bis(2,2-dimethyl-5-(sec-butylamino)-3-hexanonato)strontium(N-methylpyrrolidinone), bis(2,2-dimethyl-5-(sec-butylamino)-3-hexanonato)strontium(N,N-Diethylacetamide).

18. A vapor deposition process for forming a conformal multi-component metal oxide thin film on a substrate wherein at least two metal containing complexes and an oxygen containing agent are introduced to a deposition chamber and a multi-component metal oxide film is deposited on a substrate, the improvement which comprises using at least two metal containing complexes, having different metals, selected from: 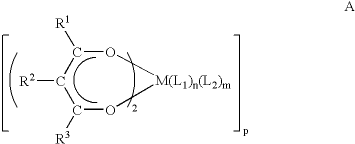
wherein M is selected from Mg, Ca, Sr, and Ba; R¹ and R³ are independently selected from the group consisting of linear or branched alkyl, alkoxyalkyl, fluoroalkyl: having from 1 to 10 carbon atoms, cycloaliphatic, and aryl having from 4 to 12 carbon atoms; R² is selected from the group consisting of hydrogen, linear or branched alkyl, fluoroalkyl, alkoxy: having from 1 to 10 carbon atoms, cycloaliphatic, and aryl: having from 4 to 12 carbon atoms; L, is selected from the group consisting of the organic amide class RCONR′R″ wherein R and R′ are linear or branched alkyl having from 1-10 carbon atoms and they can be connected to form a cyclic group (CH₂)_{q}, wherein q is from 4-6, and R″ is selected from alkyl having from 1 to 4 carbon atoms and cycloalkyl having from 4 to 8 carbon atoms; L₂ is selected from the group consisting of the organic amide class RCONR′R″ wherein R and R′ are independently linear or branched alkyl having from 1-10 carbon atoms and they can be connected to form a cyclic group (CH₂)_{q}, wherein q is from 4-6; n is a number selected from between 1 and 4; m is selected from a number between 0 to 4; p is selected from 1, 2; 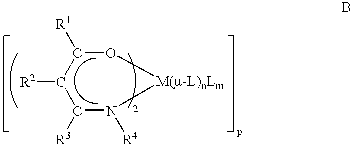
wherein M is selected from Mg, Ca, Sr, and Ba; R¹ and R⁴ are selected from the group consisting of linear or branched alkyl, alkoxyalkyl, fluoroalkyl: having from 1 to 10 carbon atoms, cycloaliphatic, and aryl: having from 4 to 12 carbon atoms; R² and R³ are selected from the group consisting of hydrogen, linear or branched alkyl, alkoxyalkyl, fluoroalkyl, alkoxy: having from 1 to 10 carbon atoms, cycloaliphatic, and aryl: having from 4 to 12 carbon atoms, L is selected from the group of the organic amide class RCONR′R″ wherein R and R′ are independently alkyl having from 1-10 carbon atoms and they can be connected to form a cyclic group (CH₂)_{q}, wherein q is from 4-6, and R″ is selected from alkyl having from 1 to 4 carbon atoms and cycloalkyl having from 4 to 8 carbon atoms; n is a number selected from between 0 and 4; m is selected from a number between 1 to 4; p is selected from 1, 2; μ-L indicates L is connected to two metals, M, via μL's oxygen atom when p=2; and, 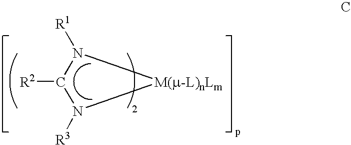
wherein M is selected from Mg, Ca, Sr, and Ba; R¹ and R³ are independently selected from the group consisting of linear or branched alkyl, alkoxyalkyl, fluoroalkyl: from 1 to 10 carbon atoms, cycloaliphatic, and aryl: having from 4 to 12 carbon atoms; R² is selected from the group consisting of hydrogen, linear or branched alkyl, fluoroalkyl, alkoxy: from 1 to 10 carbon atoms, cycloaliphatic, and aryl: having from 4 to 12 carbon atoms; L is selected from the group of the organic amide class RCONR′R″ wherein R and R′ are independently linear or branched alkyl having from 1-10 carbon atoms and they can be connected to form a cyclic group (CH₂)_{q}, wherein q is from 4-6, and R″ is selected from alkyl having from 1 to 4 carbon atoms and cycloalkyl: having from 4 to 8 carbon atoms, n is a number selected from between 0 and 4; m is selected from a number between 1 to 4; p is selected from 1, 2; μ-L indicates L is connected to two metals, M, via μL's oxygen atom when p=2.

19. The process of claim 18 wherein the vapor deposition process is selected from the group consisting of chemical vapor deposition, cyclic chemical vapor deposition and atomic layer deposition.

20. The process of claim 18 wherein the oxygen containing agent is selected from the group consisting of N₂O, O₂, H₂O₂, H₂O, ozone, O₂ plasma, organic peroxide, and mixtures thereof.

21. The process of claim 18 wherein a first metal containing complex is selected from the group consisting of Sr₂(tmhd)₄(NMP)₄, and Sr₂(tmhd)₄(DEAC)₃, and a second metal containing complex is selected from the group consisting of Ba₂(tmhd)₄(NMP)₄.(H₂O)₂, Ba₂(hfac)₄(NMP)₅.

22. The process of claim 21 wherein a third metal containing complex is selected from the group consisting of titanium containing alkoxides and beta-ketonates and mixtures thereof.

23. The process of claim 22 wherein the third metal containing complex is selected from the group consisting of Ti(OPrⁱ)₄, Ti(tmhd)₂(OPrⁱ)₂, where Prⁱ=isopropyl, where tmhd=2,2,6,6-tetramethyl-3,5-heptanedionate, Ti(mpd)(tmhd)₂, where mpd=2-methyl-2,4-pentanedioxy, Ti(4-(2-methylethoxy)imino-2-pentanoate)₂ and mixtures thereof.
